(19) United States
(12) Patent Application Publication
(10) Pub. No.: US 2015/0245988 A1
(43) Pub. Date: Sep. 3, 2015

(54) LIQUID COSMETIC AND/OR DERMOCOSMETIC COMPOSITION

(71) Applicant: Pharmaclay Delivery Systems, S.L., San Sebastian (ES)

(72) Inventors: Juan Luis GONZALEZ SEGURA, San Sebastian (ES); Lorena Arrastua Barbado, Villabona (Guipuzcoa) (ES)

(21) Appl. No.: 14/714,494

(22) Filed: May 18, 2015

Related U.S. Application Data
(63) Continuation-in-part of application No. 12/678,185, filed on Jul. 6, 2010.

(30) Foreign Application Priority Data
Sep. 14, 2007 (ES) P200702453
May 12, 2015 (EP) P201530639

(51) Int. Cl. A61K 8/81; A61K 8/97; A61K 8/365; B65D 83/14; A61Q 19/00; A61K 8/04; A61K 8/55; B65D 83/66; A61K 8/26; A61Q 5/00

(52) U.S. Cl. CPC A61K 8/8147; A61K 8/26; A61K 8/97; A61K 8/365; A61Q 5/00; A61Q 19/007; A61K 8/046; A61K 8/55; B65D 83/66; B65D 83/752; A61K 2800/87

(57) ABSTRACT

A cosmetic and/or dermocosmetic composition for its application in the treatment of skin, scalp and/or hair is disclosed. The composition is characterized for being a liquid compound that is dosed using a spray/aerosol system and for being composed by an active ingredient consisting on clay and cocoa powder, a skin beneficial compound if desired and water and wine alone or combined as disperse phase. The composition is isolated in a chamber without any contact with the propellant, suffers a distinctive maturation process and is rheologically designed for being able to fill and dose from the packing.

### CROSS-REFERENCE TO EARLIER FILED APPLICATIONS

This application claims the benefit of priority of Spanish Application No. P201530639 filed May 12, 2015 and is a continuation-in-part of U.S. patent application Ser. No. 12/678,185 filed Jul. 6, 2010 which is a 371 of PCT/ES2008/000585 filed Sep. 11, 2008, which claimed the priority of Spanish Application P200702453 filed Sep. 14, 2007, the contents of each Applications are incorporated herein by reference.

### FIELD OF THE INVENTION

The invention relates primarily to the field of cosmetic and dermatology. The present invention falls within the cosmetic and/or dermocosmetic composition for skin, scalp and/or hair treatment field. More specifically this invention relates to a mask type cosmetic and/or dermocosmetic composition (a composition that forms a continuous film when applied that is allowed to dry before being removed).

More specifically the present invention refers to a cosmetic and/or dermocosmetic composition for its external administration in a spray/aerosol system which at least contains an active compound (clay and cocoa powder alone or in combination), a continuous external dispersing phase (natural mineral spring water and/or wine), sodium polyphosphate, sodium polyacrylate, citric acid, and if wanted, another skin beneficial compound, characterized that the composition is isolated in a chamber without contact with the propellant in a double (or more) chamber aluminum monoblock container, suffers a maturation process and has specific rheological characteristics that allow the filling and empty of the composition from the container and the spreading of the liquid without draining over the skin, scalp and/or hair.

### BACKGROUND

Cosmetics are care substances used to enhance the appearance or odor of the human body while dermocosmetics are a special kind of cosmetics developed for precisely defined skin types or complaints.

The present invention refers to a mask type cosmetic and/or dermocosmetic composition for its external administration dosed in a spray/aerosol system which at least contains an active compound (clay and cocoa powder alone or in combination), a continuous external dispersing phase (natural mineral spring water and/or wine), sodium polyphosphate, sodium polyamilate, citric acid, and if wanted, another skin beneficial compound, characterized that the composition is isolated in a chamber without contact with the propellant in a double (or more) chamber aluminum monoblock container, suffers a maturation process and has specific rheological characteristics that allow the filling and empty of the composition from the container and the spreading of the liquid without draining over the skin, scalp and/or hair.

The use of clay as active compound for cosmetic and/or dermocosmetic compositions is not new US patent 2008/0081024 A1 (Beasley et al.) teaches a sprayable composition comprising kaolin and or cocoa butter as skin protectant agents. Preferred weight percent ranges greater than 50% for cocoa butter and from 4 to 20% for clay are claimed. Beasley also claims the composition is mixedcombined with the propellant in a preferable 30% weight percent. Beasley does not teach the use of sodium polyphosphate polyacrylate for preparing the dispersion.

US 2004/0208902 A1 (Gupta) is directed to compositions comprising zeolites (a clay type) that can be prepared in the form of sprayable compositions mixed with the propellant. Gupta does not even teach the use of sodium polyphosphate, sodium polyacrylate or citric acid or the maturation process of zeolite dispersions.

An article published on Cosmetic Design on 2004 teaches the use of cocoa powder on cosmetics. Nevertheless, this article does not describe any composition nor application way for those cosmetic products.

US 2002/0035161 A1 (Segura) is directed to topically applicable compositions for treating or caring skin using natural mineral water as aqueous dispersing phase. Segura also teaches the use of sodium polyacrylate as emulsifying system. Nevertheless Segura does not consider the use of clay or cocoa powder as active ingredient nor the application of the final product in a spray/aerosol system.

U.S. Pat. No. 5,034,226 A (Beck) teaches the use of sparkling wine for producing cosmetic products. The inventor does not use clay nor other products claimed in the present invention, nor even spray/aerosol technology.

US 2003/0104019 A1 (McCulloch et al.) teaches a liquid cosmetic composition comprising clay, wherein the composition further comprises a continuous external dispersing phase containing water, sodium polyphosphate and sodium polyacrylate. The concentration range of the clays claimed is out from the range of the present invention and the ratio of clay/dispersant is fixed. They don't teach a sprayable/aerosol composition,

The use of sodium polyphosphate and/or sodium polyacrylate to disperse clays in water is not even new, as demonstrated by U.S. Pat. No. 6,784,148 B2, EP 95306121 and U.S. Pat. No. 3,661,610. None of those patents concerns cosmetic and/or dermocosmetic applications. U.S. Pat. No. 6,781,448 B2 claims a cleaning composition from about 0.1 wt % to 5 wt % of clay. U.S. Pat. No. 3,661,610 is limited to the use of kaolin clays and its concentration ranges are smaller than in the present invention. Moreover, the patent mentions that once prepared, the composition is spray-dried. The spray-drying process is a method for producing a dry product from a liquid. The present invention describes a liquid composition, not a dry one.

Runge et al. (U.S. Pat. No. 6,332,482) teach a multirefillable container comprising a seal useful for personal care compositions. The container is tightly closed and the authors mention that is suitable for dispersing high or low viscous compositions. It is limited to dual chamber.

US 2010/0272821 (Segura) teaches a composition in which the compositional ranges are not claimed, even if the technology described is similar This leads the patent application incomplete.

The present invention claims a cosmetic and/or dermocosmetic compositions containing from middle/high concentrations of clay and cocoa powder: The compositions described on the previous art in which clay dispersions are prepared by the addition of sodium polyacrylate and/or sodium polyphosphate are focused in clay concentrations lower than 20 wt % in most cases. Moreover, most of the examples are limited to one clay type (mostly kaolin), while the present invention teaches the used of any clay selected from different clay families/groups. Because the composition depends on the structural, physicochemical and crystallographic properties of the selected clay, the compositions, especially at high clay concentrations, are not obvious. The maturation process is not mentioned in the previous art.

No examples of liquid dispersions with cocoa powder, sodium polyacrylate, sodium polyphosphate, citric acid and if necessary, other compound, are found. Moreover, liquid compositions based on cocoa powder dosed using a spray/aerosol system are not found in previous art. The properties and the rheology of the claimed compositions depend on the physicochemical properties of the selected cocoa powder. The maturation process is not mentioned in the previous art.

In addition the present patent teaches the synthesis of composition with an optimum theology that allows the filling and dosing the composition from a double or more chamber aluminum monoblock container that follows a spray/aerosol system. Contrary to prior art, a propellent free composition is claimed.

### SUMMARY OF INVENTION

The present invention refers a cosmetic and/or dermocosmetic composition for external administration dosed using a spray/aerosol system for the treatment of skin, scalp and/or hair. The composition is propellent free and contains an active compound consisting of clay and cocoa powder alone or in combination, natural mineral water, sodium polyphosphate, sodium polyacrylate, citric acid, and if wanted, another skin beneficial compound. The composition is distinguished for having high active compound concentrations, at least 20%. This concentration depends on the clay selected from any of the clay families/groups and the type of cocoa powder. The cosmetic/dermocosmetic composition is characterized for suffering a distinctive maturation process and for having specific rheological characteristics that allow its filling and empty from a double or more chamber aluminum monoblock container and the spreading of the liquid without draining.

### BRIEF DESCRIPTION OF THE FIGURES

The following figures form part of the present description and describe exemplary embodiments of the claimed invention. The skilled artisan will, in light of these figures and the description herein, be able to practice the invention without undue experimentation.

FIG. 1 illustrates XDR results of non-limiting examples of clays that can be used in the present invention. In particular, FIG. 1A describes the XDR of a white clay, FIG. 1B is the XDR of a red clay, FIG. 1C is the XDR of a black clay and FIG. 1D is the XDR of a ghassoul clay.

FIG. 2 illustrates the TGA results of non-limiting examples of clays that can be used in the present invention. In particular, FIG. 2A represents the results of a white clay, FIG. 2B is the TGA of a red clay, FIG. 2C concerns to a black clay and FIG. 2D is the TGA of a ghassoul clay.

FIG. 3 illustrates the increase of the composition viscosity over time occurring during maturation process.

FIG. 4 illustrates the shear thinning behavior of the compositions claimed in the present invention.

FIG. 5 illustrates the thixotropic behavior of the compositions claimed in the present invention.

FIG. 6 illustrates the hydrating efficacy, by means of the skin moisturizing index values measured after several application times, of the cosmetic and/or dermocosmetic masks claimed in the present invention.

FIG. 7 illustrates the exfoliating capacity, by means of the desquamation index measured after different application times, of a composition claimed in the present invention.

FIG. 8 illustrates the sebum absorption capacity, by means of the sebum content measured after several application times, of a composition claimed in the present invention.

FIG. 9 illustrates the sebo-regulating efficacy, by means of the percentage of surface occupied by oil measured after several application times, of a composition claimed in the present invention.

### DETAILED DESCRIPTION OF THE INVENTION

The liquid cosmetic and/or dermocosmetic composition for external administration dosed using a spray/aerosol system is applied toughly the skin, scalp and/or hair as a mask and confers to it hydrating/moisturizing, emollient, oil regulating, exfoliant, nutrient, brightening, smoothing, and/or any other cosmetic and/or dermocosmetic property. A mask is a composition that is applied through skin, scalp and/or hair and allowed to dry before being removed. It forms a continuous film through the skin, scalp and/or hair after is dried. The liquid cosmetic and/or dermocosmetic composition for external administration is stable at least for one year.

An aspect of the invention refers to a liquid cosmetic and/or dermocosmetic composition for external administration dosed using a spray/aerosol system comprising:
a) 20 to 80% by weight, based on the total weight of the composition, of at least an active compound selected from the group formed by day and cocoa powder alone or in combination,
b) 0.001 to 20% by weignt, based on the total weight of the composition, of sodium polyacrylate, and
c) 19.997 to 79.997% by weight, based on the total weight of the composition, of a continuous external dispersing phase selected from the group formed by natural mineral spring water and wine, alone or in combination.

In preferred embodiments, the composition further comprises a skin beneficial compound in an effective amount.

In further preferred embodiments, the composition also comprises:
d) 0.001 to 20% by weight, based on the total weight of the composition, of sodium polyphosphate,
e) 0.001 to 5% by weight, based on the total weight of the composition, of citric acid
   the concentration of clay used in the composition depends on the clay selected from any of the clay families/groups. The concentration of cocoa powder used in the composition depends on the cocoa powder selected and is comprised in an interval between 1 to 50 wt % with respect to the total amount of active compound.

Another aspect of the invention refers to a liquid cosmetic and/or dermocosmetic composition for external administration dosed using a spray/aerosol system, characterized that;
a) the composition comprises;
   i) 20 to 80% by weight, based on the total weight of the composition, of at least an active compound selected from the group formed by clay and cocoa powder alone or in combination
   ii) 19.997 to 79.997% by weight, based on the total weight of the composition, of a continuous external dispersing phase selected from the group formed by natural mineral spring water and wine, alone or in combination
   iii) at least one skin beneficial compound in an effective amount as needed and not limited to any specific limits,
   iv) 0.001 to 20% by weight, based on the total weight of the composition, of sodium polyphosphate,
   v) 0.001 to 20% by weight, based on the total weight of the composition, of sodium polyacrylate,
   vi) 0.001 to 5% by weight, based on the total weight of the composition, of citric acid
      the concentration of clay used in the composition depends on the clay selected from any of the clay families/groups. The concentration of cocoa powder used in the composition depends on the cocoa powder selected and is comprised in an interval between 1 to 50 wt % with respect to the total amount of active compound.

In one embodiment of the present invention clay (or different clay mixtures) is the active compound used together with natural mineral spring water, sodium polyacrylate, sodium polyphosphate and citric acid as materials for the synthesis of the liquid cosmetic and/or dermocosmetic composition capable to be dosed using spray/aerosol system.

In another embodiment of the present invention cocoa powder is the active compound used together with natural mineral spring water, sodium polyacrylate, sodium polyphosphate and citric acid to synthesize the liquid cosmetic and/or dermocosmetic composition capable to be dosed using spray/aerosol system.

In yet another embodiment of the present invention, both clay and cocoa powder are used as active compounds together with natural mineral spring water, sodium polyacrylate, sodium polyphosphate and citric acid to form the liquid cosmetic and/or dermocosmetic composition capable to be dosed using spray/aerosol system.

In yet another embodiment of the present invention clay is used as active compound together with a mixture of natural mineral spring water and wine as continuous phase, sodium polyacrylate, sodium polyphosphate and citric acid to form the liquid cosmetic and/or dermocosmetic composition capable to be dosed using spray/aerosol system.

In yet another embodiment of the present invention at least one skin beneficial compound in an effective amount as needed and not limited to any specific limits is added to clay and/or cocoa powder, natural mineral spring water and/or wine, sodium polyacrylate, sodium polyphosphate and citric acid to form the liquid cosmetic, and/or dermocosmetic composition capable to be dosed using spray/aerosol system.

As mentioned, in a preferred embodiment of the present invention day is the active compound used together with natural mineral spring water, sodium polyacrylate, sodium polyphosphate and citric acid as materials for the synthesis of the liquid cosmetic and/or dermocosmetic composition capable to be dosed using spray/aerosol system.

Clay and cocoa powder concentration of the final cosmetic and/or dermocosmetic composition is carried out following the principles of 1) the achievement of the highest clay and cocoa powder concentration possible, 2) being this maximum concentration the one at which the rheology of the composition is good enough to fill and dosed the composition from the final container.

Clays are deeply characterized in order to choose their concentration of the final composition. The structural, physicochemical and crystallographic properties are measured: Water absorption, structure, crystallography and cationic exchange capacity are the key properties necessary for choosing clay concentration. Smectite clay types for example are formulated at lower concentration than kaolin type clays. The higher the water absorption the lower the clay concentration in the final composition. Clays with high cation capacity are also used at lower concentration.

In general, clays used as active compounds in the present invention are composed of aluminum silicates selected from any of the clay families/groups; kaolinite, smectite/montmorillonite, chlorite and/or micatillite. Moreover, clays used as active compound are selected from any of the clay subfamilies; kaolin, hectorite, bentonite, illite, etc. Therefore, clays with different chemical and mineralogical contents (Al₂O₃, SiO₂ILL, K, Qz) can be used. Moreover, clays containing other mineralogical elements can be used (Mg, Ca, etc.). It is worth to mention that this different mineralogy is the responsible of the different structural, physicochemical and crystallographic properties of clays. Therefore, the combination of those structural, physicochemical and crystallographic properties determines the composition (concentration ranges) and properties of the liquid cosmetic and/or dermocosmetic composition dosed using a spray/aerosol system claimed in the present invention.

Examples of suitable clays include, but are not limited to, white clay from the family of kaolin, red clay from the family of kaolin, black clay from the family of kaolin and ghassoul or Moroccan clay from the family of smectites. Natural and non-modified clays are preferred. Synthetic and modified clays can be also used. The structural, physicochemical and crystallographic properties clays can be determined by means of several experimental procedures and are the responsible of the concentration of clay used and the final properties of the liquid cosmetic and/or dermocosmetic composition for external administration dosed using a spray/aerosol system disclosed in the present invention.

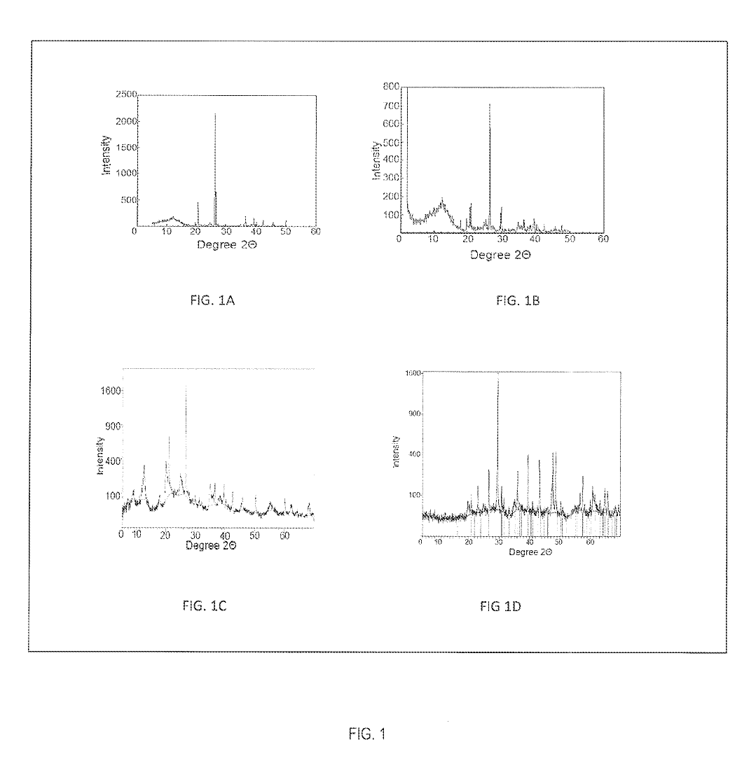

For example, the crystallography of different clays can be measured by X-ray diffraction. The X-ray diffraction occurs when the crystalline atoms cause a beam of X-rays to diffract into many specific directions. By measuring the angles and intensities of these diffracted beams, a crystallographer can produce a three-dimensional picture of the electron density within the crystal. From this electron density, the mean positions of the atoms in the crystal can be determined, as well as their chemical bonds, their disorder and other information. Examples of XDR experiments of suitable clays that are included in the invention, but not limited, are presented in FIG. 1.

From those graphs, the crystallographic composition of the clays can be determined (Table 1).

TABLE 1
Crystallographic composition of clays.

| Clay | Quartz | Other components | Kaolin | Illite |
|---|---|---|---|---|
| White Clay | 69 | — | 26 | 5 |
| Red clay | 42 | Calcite, Dolomite, | 31 | 9.5 |
| | | Alunite, Speriolite | | |
| Black clay | 29 | — | 70 | 30 |

| | | | |
|---|---|---|---|
| Ghassoul clay | 0 | Calcite, Dolomite | Smectite, but difficult to |
| | | | determine due to the low |
| | | | crystallography |

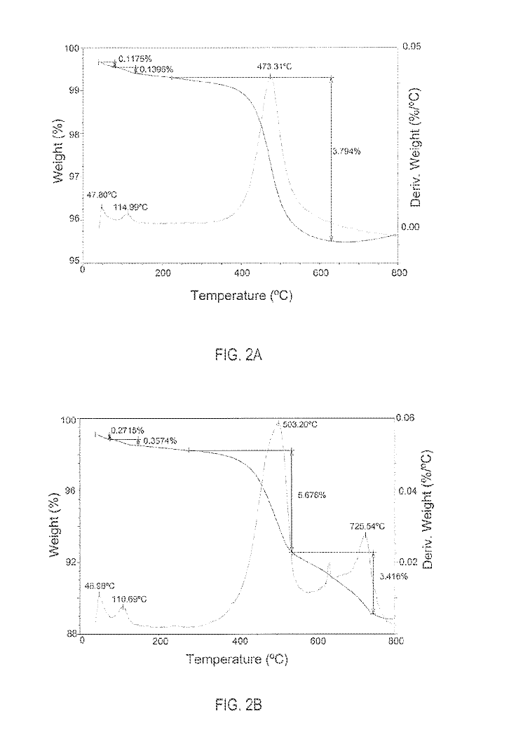

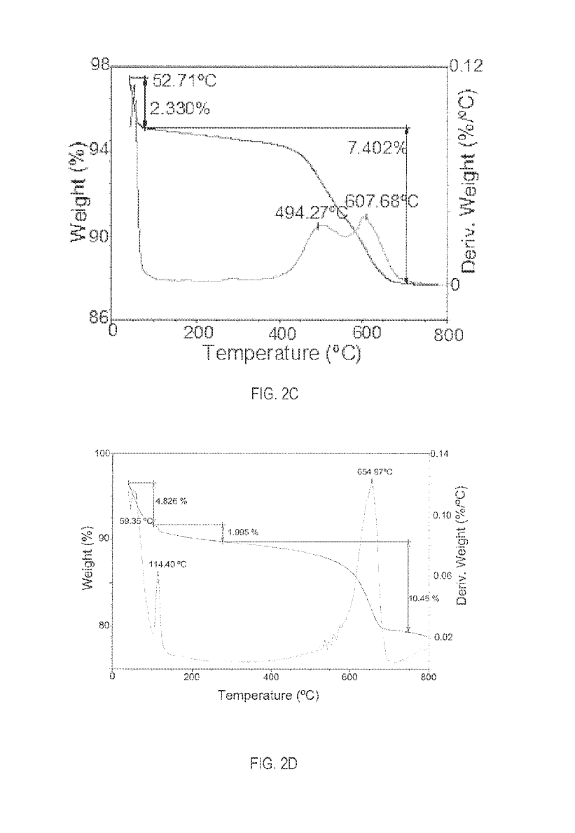

The structure of clays can be determined using Thermogravimetric Analysis (TGA). The method consists in a thermal analysis in which changes in physical and chemical properties of the materials are measured as function of the temperature or time. On clays, TGAs gives the information about dehidratation and dehidroxilation processes which indicates the structure of clay minerals. Therefore, TGA experiments also indicate the water uptake/adsorption capacity of the clays. Examples of TGA experiments of suitable clays that are included in the invention, but are not limited, are presented in FIG. 2.

The physicochemical properties of clays can be also measured. Examples of pH, conductivity and cationic exchange capacity of suitable clays that are included in the invention, but not limited, are presented in Table 2.

TABLE 2
Physicochemical properties of clays.

| | | | Cationic Exchange Capacity |
|---|---|---|---|
| Clay | pH | Conductivity (μS/cm) | (milliequivalents/100 mL) |
| White clay | 8 ± 1 | 34 ± 5 | 17 ± 4 |
| Red clay | 8 ± 2 | 119 ± 20 | 27 ± 5 |
| Black clay | 3 ± 1 | 1015 ± 20 | 37 ± 7 |
| Ghassoul clay | 7 ± 1 | 3300 ± 500 | 89 ± 22 |

The concentration of clay as active compound as well as the final properties of the liquid cosmetic and/or dermocosmetic composition for external administration dosed using a spray/aerosol system used as a mask depends on the structural, physicochemical and crystallographic properties of the selected clays (see Examples I, II, III and IV). It should be noted that raising the composition of the present invention is an innovative feature because the obtaining of dispersions with high clay concentration (as in examples II and III) is not described in the state of art. Moreover the obtaining of a hydrating/moisturizing mask based on high clay concentrations is not described in the state of art, as clay mask are known for dry the skin (Lees, M. Skin Care Beyond the Basics, Milady, 2001; Baran, R. Maibach, H. Texbook of Cosmetic Dermatology, Martin Dunitz 1994; Draelos, Z. D, Atlas of Cosmetic Dermatology, Churchill Livingstone, 2000). As comparative examples, compositions described in previous art are prepared following the composition of the present invention. US 200310419 describes the use of montmorillonite clay in a concentration of 2 wt %. US 2008/0081024A1 describes a maximum kaolin clay concentration of 20 wt % (Examples V and VI). Both dispersions based on low clay concentration are not stable as shown by the centrifugation tests (see examples). Due to their low viscosity, the compositions drain when applied to skin, scalp and/or hair. Moreover, because the low clay concentration the resulting cosmetic compositions are not masks, cosmetic and/or dermocosmetic compositions that are applied forming a continuous film and allowed to dry before being removed.

As previously mentioned, in another embodiment of the present invention cocoa powder is the active compound used together with natural mineral spring water, sodium polyacrylate, sodium polyphosphate and citric acid to form the liquid cosmetic and/or dermocosmetic composition capable to be applied using a spray/aerosol system. Moreover, in yet another embodiment of the present invention, both clay and cocoa powder mixtures are used as active compounds together with natural mineral spring water, sodium polyacrylate, sodium polyphosphate and citric acid.

In general, the cocoa derivative used in the present invention is a cocoa powder. Cocoa powder is the low fat component of chocolate while cocoa butter is composed by the fatty component. The cocoa powder used can also contain certain amount of cocoa butter. In a preferred embodiment of the present invention the cocoa powder used is alkalized and can have different concentrations of cocoa butter.

Cocoa powder is also deeply characterized in order to choose its final concentration on the composition. Water absorption is the key parameter. The higher the water absorption the lower the concentration of cocoa powder in the final composition.

The concentration of cocoa powder as active compound of the liquid cosmetic and/or dermocosmetic composition for external administration dosed using a spray/aerosol system depends on the physicochemical properties of the selected cocoa powder (see examples VIII, IX, X and XI). Namely, the concentration of cocoa powder used in the composition depends on the physicochemical properties of the selected cocoa powder.

Examples of suitable cocoa powders include, but are not limited, to cocoa powders with the characteristics of Table 3.

TABLE 3
Physicochemical properties of cocoa powder.

| Cocoa | Moisture | Total fat content on | | Conductivity |
|---|---|---|---|---|
| powder | (%) | dry matter (%) | pH | (μS/cm) |
| Sample 1 | 2.5 ± 0.5 | 10-12 | 6.0 ± 0.2 | 3200 ± 10 |
| Sample 2 | 2.0 ± 0.3 | 20-22 | 7.8 ± 0.4 | 5010 ± 40 |
| Sample 3 | 2.6 ± 0.6 | 10-12 | 8.0 ± 0.4 | 3950 ± 35 |
| Sample 4 | 1.1 ± 0.5 | 20-22 | 7.5 ± 0.2 | 4820 ± 20 |
| Sample 5 | 5.90 ± 1.9 | 11-13 | 6.0 ± 0.3 | 3180 ± 15 |

As previously mentioned, in yet another embodiment of the present invention clay is used as active compound together with a mixture of natural mineral spring water and wine as continuous phase, sodium polyacrylate, sodium polyphosphate and citric acid to form the liquid cosmetic and/or dermocosmetic composition capable to be applied using a spray/aerosol system and used as a mask

In general, the selected water is hypoallergenic and free of preservatives. Natural mineral waters from different sources can be used. Non limiting examples of natural mineral waters available to use in the present invention are the ones coming from Elkai spring, Teleno spring and Belnature spring. As non-limiting example, this water is collected in a sterile room without human operation, directly from the spring at 100 meters deep in the heart of an aquifer from a protected karstic area.

The selection of water in the liquid cosmetic and/or dermocosmetic composition for skin care is fundamental because: 1) the mineralogy of water can add synergistic effects to the overall compositions. The nature of the ions present in water can enhance the skin care contribution of the overall composition; 2) the stability of the composition changes depending on the water used as continuous phase; and 3) the rheology of the final composition can also vary depending on the water used.

In general the wine used in the present invention is a red wine. As a non-limiting example, the selected red wine has the following physicochemical characteristics:
pH=3.5±0.6
Conductivity (μS/cm)=2340±20
Dry residue (g/L)=18.5±0.4

Example XII presents a non-limited example of a composition comprising a mixture of natural mineral spring water and wine as continuous phase.

In general, sodium polyacrylate is a crosslinked acrylic type polymer, so-called, hydrogel. Is a sodium salt of a crosslinked acrylic acid polymer (the monomeric unit is (—CH₂—CH(CO₂Na)—) that is partially neutralized (50-70% of the acid groups have been converted into the respective sodium salt). The main characteristics of sodium polyacrylates are: 1) the crosslinking capacity by means of covalent bonds and/or hydrogen bondings; 2) the water adsorption capacity, as it's able to retain up to 800 times their weight in water and; 3) the swelling capability, consequence of the already mentioned characteristics.

Sodium polyacrylate is a known dispersion agent for clays. The roll of sodium polyacrylate in the present invention is to 1) help the dispersion of the active compounds of the composition and 2) give other characteristics to the liquid cosmetic and/or dermocosmetic composition. Those characteristics are:
Contribute to the desired specific rheological characteristics that allow the filling and empty of the composition from the double (or more) chamber aluminum monoblock container and the spreading without draining of the liquid over the skin scalp and/or hair.
Add an additional moisturizing/hydrating effect to the liquid composition that is applied thought the skin, scalp and/or hair (Example XIII)
Avoid the cold feeling in the skin caused by fast water evaporation from the wet film.
Maintain the original humidity of the composition even when a part of the composition is in contact with air at the exit of the double (or more) chamber aluminum monoblock container.

The additional moisturizing/hydrating effect of the final cosmetic and/or dermocosmetic composition is a synergistic effect achieved by the addition of sodium polyacrylate. The use of high clay concentration masks for moisturizing/hydrating skin, scalp and/or hair is not obvious for a skilled artisan, as previous art describes that clay masks overdry the skin, scalp and/or hair, especially when dehydrated skin is treated. (Lees, M. Skin Care Beyond the Basics, Milady, 2001; Baran, R. Maibach, H. Texbook of Cosmetic Dermatology, Martin Dunitz 1994; Draeios, Z. D. Atlas of Cosmetic Dermatology, Churchill Livingstone, 2000). Cosmetic compositions with hydrating effect that may contain clays present in previous art are always formulated with low clay concentrations and high concentrations of hydrating ingredients. More than masks are creams, lotions or serums. The present invention claims a ready to use high clay concentration mask that in combination with sodium polyacrylate is able to moisturize/hydrating the skin, scalp and/or hair.

The present invention also claims a liquid cosmetic and/or dermocosmetic composition for external administration dosed using a spray/aerosol system characterized for having the components described above and, at least, another skin beneficial compound in an effective amount as needed and not limited to any specific limits.

Exemplary of such skin beneficial compounds may be conditioners, secondary emollient or hydrating agents, additives which enhance appearance or fragrance, preservatives, anti-microbial compounds, fungicides, skin conditioners, absorbents, hair fixing agents, smoothing agents, soothing agents, anti-inflammatory agents, skin lightening or whitening products, anti-oxidants, anti-aging compounds, skin protecting agents, anti-wrinkle compounds, acne treatment compounds, body slimmings, and any other compound that can be beneficial to the skin.

Examples of those skin beneficial compounds include but are not limited to lavender essential oil, bergamot essential oil, orange essential oil, aloe vera extract, ginseng extract, argan oil, Andean quillaja saponaria extract, licorice extract, green tea extract, macadamia oil, algae extract, harpagophytum extract, menthol, camphor and methyl salicylate, alone or in combination.

Examples of compositions containing these skin beneficial compounds can be found in Examples XIII, XIV and XV.

The liquid cosmetic and/or dermocosmetic composition for external administration dosed using a spray/aerosol system of the present invention is further characterized because is isolated in a chamber without contact with the propellant in a double (or more) chamber aluminum monoblock container. This container ensures the purity and microbiological stability of the composition as the product doesn't have any contact nor with propellant (is isolated) nor with ambient air.

This packing allows a long-term stability and drying up resistance, makes possible the use of the composition at any moment without previous elaboration and avoids microbiological contamination.

In a preferred embodiment of the present invention the composition is isolated in a chamber without contact with the propellant in a double chamber aluminum monoblock container that follows piston technology.

In another preferred embodiment of the present invention the composition is isolated in a chamber without contact with the propellant in a double chamber aluminum monoblock container that follows bag-on-valve technology.

Non-limiting examples of propellants used in the present invention are nitrogen and air. Preferentially, air, an inert and ecological gas, respectful with the product, that preserves at any moment its microbiological purity and freshness, is used as propellant.

In general the liquid cosmetic and/or dermocosmetic composition for external administration dosed using spray/aerosol technology claimed in the present invention suffers a maturation process. The maturation process is a mixture of physicochemical processes in which a three dimensional structure is formed inside the composition. The maturation process is a process that occurs over time until equilibrium is reached and varies from days to months. The maturation process comes from; 1) the interactions created between the clay platelets and/or cocoa powder and external dispersing water phase (due to cationic exchange and swelling processes); 2) the interactions between the sodium polyacrylate chains and the external dispersing water phase (due to hydrogen bonding) and; 3) the interaction between the active compounds (clay and/or cocoa powder) with the sodium polyacrylate chains and the external dispersing water phase. The resultant composition is a tree dimensional structured network with modified properties in comparison to the freshly prepared composition. The cationic exchange between clay and/or cocoa powder and the external dispersion water phase makes the ions and minerals redistribute, changing the pH and the conductivity of the freshly prepared composition over time until equilibrium is reached. Redistribution of ions and minerals might also increase the therapeutic use of the composition, as clays are rich on ions/minerals. The increase of interactions between sodium polyacrylate, water and clay and/or cocoa powder, as well as the swelling of clay, cocoa powder and sodium polyacrylate leads also to a redistribution of the components during the maturation process, changing the viscosity (rheology) and the density of the composition. A non-limiting example of how the viscosity changes during maturation process until equilibrium is reached is presented in Example XVI.

The liquid cosmetic and/or dermocosmetic composition claimed in the present invention presents specific rheological characteristics that allow the filling and empty of the composition from the double (or more) chamber aluminum monoblock container and the spreading of the liquid without draining. The desired specific rheological characteristic in the present invention are:
Shear thinning properties (Example XVII): Because the present composition is isolated in a double (or more) chamber aluminum monoblock container its necessary that the viscosity of the product during dosage decreases to allow container empty. Moreover, in a non-limiting example of the present invention, the technology used as double chamber aluminum monoblock container is a bag-on-valve technology in which the composition is injected thought a valve. As consequence, it's also necessary that the viscosity of the composition decreases during container filling.
Thixotropy (Example XVIII). Thixotropy is the property of certain materials that are able to recover their original viscosity and texture after a stress suffered is removed. Because the present composition is isolated in a double (or more) chamber aluminum monoblock container it's necessary that the viscosity of the product during dosage decreases (shear thickening effect) but it's also necessary that the product recovers its original viscosity in order to avoid liquid draining through the skin, scalp and/or hair and to obtain a good spreading.

In addition to the hydrating/moisturizing effect, the liquid cosmetic and/or dermocosmetic composition for external administration dosed using a spray/aerosol system is applied thought the skin, scalp and/or hair provides a benefit to the application zone. The benefits given by the composition depends, for example, on the clay selected from the different clay families, as their properties in skin care depend on their crystallographic, structural and physicochemical properties. It also depends on the cocoa powder, water or wine used, as well as in the selected skin beneficial compound (if added). Not limiting examples of the benefits of two compositions with different active principles are presented in Examples XX and XXI.

### EXAMPLE I

### Cosmetic Composition in Spray Using White Clay as Active Compound

The white clay was first extracted from a selected mine, grinded using a dry mill method and finally sieved in order to obtain a clay powder with a particle size lower than 150 μm. Each of the components was then weighted following the formulation given in the table below (Table 4).

TABLE 4
Composition: white clay as active compound

| | wt % (weight based on the total |
|---|---|
| Compound | weight of the composition) |
| White clay | 69.40 |
| Sodium polyacrylate (dissolution) | 0.06 |
| Sodium polyphosphate (dissolution) | 0.44 |
| Citric acid (dissolution) | 0.05 |
| Natural Mineral Spring Water | 30.05 |

The emulsion was prepared using a Ultra-turrax equipment at 800 rpm for 15 minutes. The bulk emulsion was then dosed into the final recyclable double chamber aluminum monoblock container. One of those two chambers was only filled with the bulk product. The other chamber was filled with air, used as propellant. The main characteristic of this double chamber aluminum monoblock container is that the product and the propellant never get in contact, neither during storage nor lust prior to use.

The monoblock container was further sterilized by means of electron beam radiation (beta rays) in order to achieve a microorganism free final cosmetic product in a spray form.

The final properties of the cosmetic product in spray are presented in Table 5.

TABLE 5
Properties: white clay as active compound

| Color | White-grey |
|---|---|
| Texture | Bright viscous emulsion |
| pH | 6.22 |
| Viscosity (mPa · s) (10 rpm, Fungilab spindle PF) | 310000 ± 80000 |

### EXAMPLE II

### Cosmetic Composition in Spray Using a Mixture of White and Red Clays as Active Compounds

The white and red clays were first extracted from a selected mine, grinded using a thy mill method and finally sieved in order to obtain a clay powder with a particle size lower than 150 μm. Each of the components was then weighted following the formulation given in the table below (Table 6).

TABLE 6
Composition: mixture of white and red clays as active compounds.

| | wt % (weight based on the total |
|---|---|
| Compound | weight of the composition) |
| White clay | 33.07 |
| Red clay | 33.07 |
| Sodium polyacrylate (dissolution) | 0.085 |
| Sodium polyphosphate (dissolution) | 0.65 |
| Citric acid (dissolution) | 0.08 |
| Natural Mineral Spring Water | 33.07 |

The emulsion was prepared using a Ultra-turrax equipment at 800 rpm for 15 minutes and then dosed into the final recyclable double chamber aluminum monoblock container. One of those two chambers was only filled with the bulk product. The other chamber was filled with air, used as propellant. The main characteristic of this double chamber aluminum monoblock container is that the product and the propellant never get in contact, not during storage nor just prior to use.

The monoblock container was further sterilized by means of electron beam radiation (beta rays) in order to achieve a microorganism free final cosmetic product in a spray form.

The final properties of the cosmetic: product in spray are presented in the following table (Table 7).

TABLE 7
Properties: mixture of white and red clays as active compounds.

| Color | Red emulsion |
|---|---|
| Texture | Bright viscous emulsion |
| pH | 6.68 |
| Viscosity (mPa · s) (10 rpm, Fungilab spindle PE) | 140000 ± 800 |

### EXAMPLE III

### Cosmetic Composition in Spray Using a Black Clay as Active Compound

The black clay first extracted from a selected mine, grinded using a dry mill method and finally sieved in order to obtain a clay powder with a particle size lower than 150 μm. Each of the components was then weighted following the formulation given in the Table 8.

TABLE 8
Composition: black clay as active compound.

| | wt % (weight based on the total |
|---|---|
| Compound | weight of the composition) |
| Black clay | 63.0 |
| Sodium polyacrylate (dissolution) | 0.50 |
| Sodium polyphosphate (dissolution) | 1.50 |
| Citric acid (dissolution) | 0.10 |
| Natural Mineral Spring Water | 34.90 |

The bulk emulsion was prepared using a Ultra-turrax equipment at 800 rpm for 15 minutes and dosed into the final recyclable double chamber aluminum monoblock container. One of those two chambers was only filled with the bulk product. The other chamber was filled with air, used as propellant. The main characteristic of this double chamber aluminum monoblock container is that the product and the propellant never get in contact, not during storage nor just prior to use.

The monoblock container was further sterilized by means of electron beam radiation (beta rays) in order to achieve a microorganism free final cosmetic product in a spray form.

The final properties of the cosmetic product in spray are presented in Table 9.

TABLE 9
Properties: black clay as active compound.

| Color | Black emulsion |
|---|---|
| Texture | Bright viscous emulsion |
| pH | 5.0 |
| Viscosity (mPa · s) (10 rpm, Fungilab spindle PE) | 224000 ± 18000 |

### EXAMPLE IV

### Cosmetic Composition in Spray Using a Ghassoul Clay Active Compound

The ghassoul clay was first extracted from a selected mine, grinded using a dry mill method and finally sieved in order to obtain a clay powder with a particle size lower than 150 μm. Each of the components was then weighted following the formulation given in the table below (Table 10).

TABLE 10
Composition: ghassoul clay as active compound.

| | wt % (weight based on the total |
|---|---|
| Compound | weight of the composition) |
| Ghassoul clay | 40.00 |
| Sodium polyacrylate (dissolution) | 0.38 |
| Sodium polyphosphate (dissolution) | 20.00 |
| Citric acid (dissolution) | 0.01 |
| Natural Mineral Spring Water | 39.61 |

The bulk emulsion was prepared using a Ultra-turrax equipment at 800 rpm for 15 minutes and dosed into the final recyclable double chamber aluminum monoblock container. One of those two chambers was only filled with the bulk product. The other chamber was filled with air, used as propellant. The main characteristic of this double chamber aluminum monoblock container is that the product and the propellant never get in contact, not during storage nor just prior to use.

The monoblock container was further sterilized by means of electron beam radiation (beta rays) in order to achieve a microorganism free final cosmetic product in a spray form.

The final properties of the cosmetic product in spray are presented in the following table (Table 11):

TABLE 11
Properties: ghassoul clay as active compound.

| Color | Brown |
|---|---|
| Texture | Bright viscous emulsion |
| pH | 5.99 |
| Viscosity (mPa · s) (10 rpm, Fungilab spindle PE) | 294000 ± 27000 |

As demonstrated by the examples, the concentration of components and the final physicochemical properties of the composition depend on the selected clay and varies from clay to clay. Therefore, its important to point out that the composition disclosed in the present invention is not obvious. Moreover, in comparison to prior art, medium and high clay concentrations of clay are used.

### EXAMPLE V

### Cosmetic Composition Based on US 2003/010419

US 2003/010419 describes a liquid cosmetic composition comprising clay (montmorillonite clay at 2 wt %), water, sodium polyphosphate and sodium polyacrylate. It does not teach a sprayable composition nor a mask. A composition following US 2003/01419 clay concentration and the components of the present invention was prepared. Each of the components were weighted following the formulation given in the table below (Table 12, based on the composition of Example I).

TABLE 12
Composition: based on US 2003/010419

| | wt % (weight based on the total |
|---|---|
| Compound | weight of the composition) |
| Montmorillonite | 2.00 |
| Sodium polyacrylate (dissolution) | 0.06 |
| Sodium polyphosphate (dissolution) | 0.44 |
| Citric acid (dissolution) | 0.05 |
| Natural Mineral Spring Water | 97.45 |

The emulsion was prepared using a Ultra-turrax equipment at 800 rpm for 15 minutes: The final composition was a very low clay content dispersion with the following properties ( ).

TABLE 13
Properties: based on US 2003/010419

| Color | Beige |
|---|---|
| Texture | See-through liquid with a sediment |
| pH | 7.25 |
| Viscosity (mPa · s) | 477 ± 68 |
| (10 rpm, Fungilab spindle PA) | |
| Density (g/mL) | 1.0 ± 0.1 |
| Centrifugation test (%) | 85 ± 1 |

The composition formulated following US 2003/01419 is not a clay mask because due to the high water concentration is unable to form a continuous film that is allowed to dry and removed afterwards. The compositions formed in the present inventions are clay and/or cocoa powder masks that form continuous films thought the skin, scalp and/or hair; after drying the clay and/or cocoa powder remain in the applied surface forming a continuous film. The composition of the previous ad is not stable; sedimentation of components, especially clay, occurs in less than 5 minutes after synthesis. The centrifugation test, an indirect measurement of emulsion stability, is also very high in the composition based on previous art example. The higher the centrifugation test %, the lower the emulsion stability. The centrifugation test values of the cosmetic and/or dermocosmetic mask claimed in the present invention range from 0.1 to 10%. Claimed compositions are stable for at least two years at ambient temperature. Therefore, as innovation in comparison to prior art, the claimed composition has an improved final application ability (because is a mask) and is stable. The composition claimed in the present invention is also designed for presenting specific rheological characteristics that allow the filling and emptying of the composition from the aluminum monoblock container having two or more chambers and the spreading of the liquid without draining. Due to its low viscosity, the composition based on prior ad drains through the skin, scalp and/or hair when is applied.

### EXAMPLE VI

### Cosmetic Composition Based on US 2008/0081024 A1

US 2008/0081024 A1 describes a liquid cosmetic composition comprising kaolin (kaolin type clay in a maximum of 20 wt %) and water. A composition following US 2008/0081024 A1 clay concentration and the components of the present invention was prepared. Each of the components was than following the formulation given in the table below ( ) and based on the composition of Example I.

TABLE 14
Composition: based on US 2008/0081024 A1

| | wt % (weight based on the total |
|---|---|
| Compound | weight of the composition) |
| Kaolin | 20.00 |
| Sodium polyacrylate (dissolution) | 0.06 |
| Sodium polyphosphate (dissolution) | 0.44 |
| Citric acid (dissolution) | 0.05 |
| Natural Mineral Spring Water | 79.45 |

The emulsion was prepared using a Ultra-turrax equipment at 800 rpm for 15 minutes. The final composition was a low clay content emulsion with the following properties (

Table 15).

TABLE 15
Properties: based on US 2008/0081024 A1

| Color | Grey |
|---|---|
| Texture | See-through liquid with a sediment |
| PH | 5.19 |
| Viscosity (mPa · s) | 150 ± 20 |
| (10 rpm, Fungilab spindle PA) | |
| Density (g/mL) | 1.1 ± 0.1 |
| Centrifugation test (%) | 70 ± 1 |

The composition based on US 2008/0081024 A1 is unstable; sedimentation occurs after 5 minutes of synthesis and the centrifugation test is also so high. Because the high water concentration, the viscosity and density of the composition are too low and it drains when is applied to skin, scalp and/or hair. The composition is not even a mask, as it does not create a continuous film thought the application surface that need to be removed once its dry. The present invention claims a high clay content hydrating/moisturizing mask stable for at least two years that does not drain when is applied to skin, scalp and/or hair.

### EXAMPLE VII

### Cosmetic Composition in Spray Using Cocoa Powder Sample 1 as Active Compound

In the present example Sample 1 cocoa powder was selected as active compound. This active compound was first dry milled in order to achieve the seed powder of the desired particle size The composition is shown in Table 16

TABLE 16
Composition: cocoa powder Sample 1 as active compound.

| | wt % (weight based on the total |
|---|---|
| Compound | weight of the composition) |
| Cocoa Powder Sample 1 | 33.33 |
| Sodium polyacrylate (solution) | 0.11 |
| Sodium polyphosphate (solution) | 0.78 |
| Citric acid (solution) | 0.01 |
| Natural Mineral Water | 65.76 |

The final emulsion was prepared at ambient temperature using an Ultra-turrax equipment working for 15 minutes at 800 rpms and dosed into the foal recyclable double chamber aluminum monoblock container having air as propellant. The final properties of the cosmetic product in spray are presented in Table 17.

TABLE 17
Properties: cocoa powder Sample 1 as active compound.

| Color | Brown |
|---|---|
| Texture | Bright viscous emulsion |
| pH | 5.79 |
| Viscosity (mPa · s) (10 rpm, Fungilab spindle PE ) | 56000 ± 11000 |

### EXAMPLE VIII

### Cosmetic Composition in Spray Using Cocoa Powder Sample 2 as Active Compound

In the present example Sample 2 cocoa powder was selected as active compound. This active compound was first dry milled in order to achieve the seed powder of the desired particle size. The composition is presented in Table 18.

TABLE 18
Composition: cocoa powder Sample 2 as active compound.

| | wt % (weight based on the total |
|---|---|
| Compound | weight of the composition) |
| Cocoa Powder Sample 2 | 33.00 |
| Sodium polyacrylate (solution) | 0.11 |
| Sodium polyphosphate (solution) | 0.78 |
| Citric acid (solution) | 0.02 |
| Natural Mineral Water | 66.09 |

The final emulsion was prepared at ambient temperature using an Ultra-turrax equipment working for 15 minutes at 800 rpm and dosed into the final recyclable doable chamber aluminum monoblock container having air as propellant. The final properties of the cosmetic product in spray are presented in the following table (Table 19).

TABLE 19
Properties: cocoa powder Sample 2 as active compound.

| Color | Dark brown |
|---|---|
| Texture | Bright viscous emulsion |
| pH | 7.7 |
| Viscosity (mPa.s) (10 rpm, Fungilab spindle PE) | 78000 ± 5000 |

### EXAMPLE IX

### Cosmetic Composition in Spray Using Cocoa Powder Sample 3 as Active Compound

In the present example Sample 3 cocoa powder was selected as active compound. This active compound was first dry milled in order to achieve the seed powder of the desired particle size. The composition is presented in Table 20.

TABLE 20
Composition: cocoa powder Sample 3 as active compound.

| | wt % (weight based on the |
|---|---|
| Compound | total weight of the composition) |
| Cocoa Powder Sample 3 | 34.17 |
| Sodium polyacrylate (solution) | 0.11 |
| Sodium polyphosphate (solution) | 0.81 |
| Citric acid (solution) | 0.02 |
| Natural Mineral Water | 64.89 |

The emulsion was prepared at ambient temperature using an Ultra-turrax equipment working for 15 minutes at 800 rpms and dosed into the final recyclable double chamber aluminum monoblock container having air as propellant. The final properties of the cosmetic product in spray are presented in Table 21.

TABLE 21
Properties: cocoa powder Sample 3 as active compound.

| Color | Brown |
|---|---|
| Texture | Bright viscous emulsion |
| pH | 7.54 |
| Viscosity (mPa.s) (10 rpm, Fungilab spindle PE) | 56000 ± 2000 |

### EXAMPLE X

### Cosmetic Compost in Spray Using Cocoa Powder Sample 4 as Active Compound

In the present example Sample 4 cocoa powder was selected as active compound. This active compound was first dry milled in order to achieve the seed powder of the desired particle size.

TABLE 22
Composition: cocoa powder Sample 4 as active compound.

| | wt % (weight based on the total |
|---|---|
| Compound | weight of the composition) |
| Cocoa Powder Sample 4 | 33.34 |
| Sodium polyacrylate (solution) | 0.11 |
| Sodium polyphosphate (solution) | 0.77 |
| Citric acid (solution) | 0.01 |
| Natural Mineral Water | 65.77 |

The emulsion was prepared at ambient temperature using an Ultra-turrax equipment working for 15 minutes at 800 rpms and dosed into the final recyclable double chamber aluminum monoblock container having air as propellant. The final properties of the cosmetic product in spray are presented in the following table (Table 23).

TABLE 23
Properties: cocoa powder Sample 4 as active compound.

| Color | Dark brown |
|---|---|
| Texture | Bright viscous emulsion |
| pH | 7.18 |
| Viscosity (mPa.s) (10 rpm, Fungilab spindle PE) | 105000 ± 11000 |

### EXAMPLE XI

### Cosmetic Composition in Spray Using Cocoa Powder Sample 5 as Active Compound

In the present example Sample 5 cocoa powder was selected as active compound. This active compound was first dry milled in order to achieve the seed powder of the desired particle size.

TABLE 24
Composition: cocoa powder Sample 5 as active compound.

| | wt % (weight based on the |
|---|---|
| Compound | total weight of the composition) |
| Cocoa Powder Sample 5 | 39.80 |
| Sodium polyacrylate (solution) | 0.13 |
| Sodium polyphosphate (solution) | 0.73 |
| Citric acid (solution) | 0.01 |
| Natural Mineral Water | 59.33 |

The final emulsion was prepared at ambient temperature using an Ultra-turrax equipment working for 15 minutes at 800 rpm and dosed into the final recyclable double chamber aluminum monoblock container having nitrogen as propellant.

The final properties of the cosmetic product in spray are presented in the following table (Table 25).

TABLE 25
Properties: cocoa powder Sample 5 as active compound.

| Color | Dark brown |
|---|---|
| Texture | Bright viscous emulsion |
| pH | 5.3 |
| Viscosity (mPa.s) (10 rpm, Fungilab spindle PE) | 150000 ± 10000 |

The examples presented above indicate that the physicochemical and rheological properties of the liquid cosmetic and/or dermocosmetic compositions synthesized using different cocoa powders as active compounds depend on the type of cocoa powder used. This remarks the fact that the present invention has innovative character. Moreover, its important to point out that in prior art, cocoa butter is mostly used in skin care applications.

### EXAMPLE XII

### Cosmetic Composition in Spray Using Red Clay as Active Compound and Natural Mineral Spring Water and Wine as Continuous Media

In the present example red clay was selected as active compound. This active compound was first dry milled in order to achieve the seed powder of the desired particle size. The composition is presented in Table 25.

TABLE 26
Composition: water and wine as continuous media.

| | wt % (weight based on the total |
|---|---|
| Compound | weight of the composition) |
| Red clay | 65.43 |
| Wine | 7.46 |
| Sodium polyacrylate (solution) | 0.14 |
| Sodium polyphosphate (solution) | 2.66 |
| Citric acid (solution) | 0.07 |
| Natural Mineral Water | 24.24 |

The emulsion was prepared at ambient temperature using an Ultra-turrax equipment working for 15 minutes at 800 rpms and dosed into the final recyclable double chamber aluminum monoblock container having air as propellant. The final properties of the cosmetic product in spray are presented in the following table (Table 27).

TABLE 27
Properties: water and wine as continuous media.

| Color | Red |
|---|---|
| Texture | Bright viscous emulsion |
| pH | 6.06 |
| Viscosity (mPa.s) (10 rpm, Fungilab spindle PE) | 199000 ± 25000 |

### EXAMPLE XIII

### Cosmetic Composition in Spray Using White Clay as Active Compound and Natural Mineral Spring Water and Lavender Essential Oil as Skin Beneficial Compound

In the present example white clay was selected as active compound. This active compound was first dry milled in order to achieve the seed powder of the desired particle size. The composition is presented in

TABLE 28
Composition: addition of lavender essential skin beneficial agent.

| | wt % (weight based on the total |
|---|---|
| Compound | weight of the composition) |
| White clay | 68.00 |
| Lavender essential oil | 0.30 |
| Sodium polyacrylate (solution) | 0.20 |
| Sodium polyphosphate (solution) | 0.57 |
| Citric acid (solution) | 0.05 |
| Natural Mineral Water | 30.88 |

The final emulsion was prepared at ambient temperature using an Ultra-turrax equipment working for 15 minutes at 800 rpms and dosed into the final recyclable double chamber aluminum monoblock container having air as propellant. The final properties of the cosmetic product in spray are presented in the following table (Table 29).

TABLE 29
Properties: addition of lavender essential oil as skin beneficial agent

| Color | White-grey |
|---|---|
| Texture | Bright viscous emulsion |
| pH | 6.14 |
| Viscosity (mPa.s) (10 rpm, Fungilab spindle PE) | 180000 ± 30000 |

### EXAMPLE XIV

### Cosmetic Composition Spray Using White Clay as Active Compound and Natural Mineral Spring Water and Aloe Vera and Orange Essential Oil as Skin Beneficial Compounds

In the present example white clay was selected as active compound. This active compound was first thy milled in order to achieve the seed powder of the desired particle size. The composition is presented in Table 30.

TABLE 30
Composition: addition of aloe vera and orange
essential oil as skin beneficial agents.

| | wt % (weight based on the total |
|---|---|
| Compound | weight of the composition) |
| White clay | 66.8 |
| Aloe vera extract | 0.12 |
| Orange essential oil | 0.04 |
| Sodium polyacrylate (solution) | 0.07 |
| Sodium polyphosphate (solution) | 0.25 |
| Citric acid (solution) | 0.03 |
| Natural Mineral Water | 32.69 |

The final emulsion was prepared at ambient temperature using an Ultra-turrax equipment working for 15 minutes at 800 rpm and dosed into the final recyclable double chamber aluminum monoblock container having air as propellant. The final properties of the cosmetic product in spray are presented in the following table (Table 31).

TABLE 31
Properties: addition of aloe vera and orange
essential oil as skin beneficial agents.

| Color | White-grey |
|---|---|
| Texture | Bright viscous emulsion |
| PH | 6.46 |
| Viscosity (mPa.s) (10 rpm, Fungilab spindle PE) | 103000 ± 10000 |

### EXAMPLE XV

### Cosmetic Composition in Spray Using White Clay as Active Compound and Natural Mineral Spring Water and Ginseng Extract as Skin Beneficial Compound

In the present example white clay was selected as active compound. This active compound was first dry milled in order to achieve the seed powder of the desired particle size. The composition is presented in Table 32.

TABLE 32
Composition: addition of ginseng extract as skin beneficial agent.

| | wt % (weight based on the total |
|---|---|
| Compound | weight of the composition) |
| White clay | 68.40 |
| Ginseng extract | 0.74 |
| Sodium polyacrylate (solution) | 0.05 |
| Sodium polyphosphate (solution) | 0.53 |
| Citric acid (solution) | 0.05 |
| Natural Mineral Water | 30.23 |

The emulsion was prepared at ambient temperature using an Ultra-turrax equipment working for 15 minutes at 800 mass and dosed into the final recyclable double chamber aluminum monoblock container having air as propellant. The final properties of the cosmetic product in spray are presented in the following table (

Table 33).

TABLE 33
Properties: addition of ginseng extract as skin beneficial agent

| Color | White-grey |
|---|---|
| Texture | Bright viscous emulsion |
| pH | 6.00 |
| Viscosity (mPa.s) (10 rpm, Fungilab spindle PE) | 206000 ± 10000 |

### EXAMPLE XVI

### Maturation Process of the Composition of Example I

The maturation process of the liquid composition is one of the main characteristics of the composition claimed in the present invention. Maturation is a complex process that, in general, involves water, clay and the ions and minerals that are contained in them. In the present patent sodium polyacrylate also takes part in the maturation process. The process takes place over time (from days to months, depending on the selected clay) and during this period the properties of the composition changes till equilibrium is reached. During maturation process water is inserted between clay platelets increasing its interlaminar distance and making the clay to expand. Moreover, the ions and minerals dissolved in water phase are exchanged with the ions present on clay surface. At the same time sodium polyacrylate is swelled by water and, finally, hydrogen bonding is formed between water, clay and sodium polyacrylate. All the changes on the ion and mineral distribution make the pH and conductivity change over the time maturation occurs. Moreover, changes on clay platelets distribution, mostly expansion, and sodium polyacrylate swelling process increases the interactions between clay platelets and clay-polyacrylate, increasing composition viscosity. Water content is related with heat retention capacity of clays; the higher the water content between clay platelets, the higher the heat capacity. Therefore, maturation process, improves some physical properties of the clay minerals, such as heat retention capacity, rheology, and adhesion. At the same time, changes in mineralogy (mineral distribution) allow clay minerals to be dissolved on aqueous phase. These modifications lead to the therapeutic effect improvement of the final composition.

The maturation process takes place under the following conditions: the composition is synthesized and placed in a double or more chamber monoblock aluminum container. A sample of the freshly prepared composition is taken and the physicochemical characterization is carried out. The containers are placed in a dry and fresh place to allow the physicochemical processes to occur till equilibrium is reached. The maturation process is monitored by taking samples at different day periods and measuring the physicochemical properties of the samples; pH, conductivity, density, viscosity and centrifugation test. Due to processes as ion and mineral exchange, swelling and hydrogen bonding the physicochemical parameters change over time till equilibrium is reached.

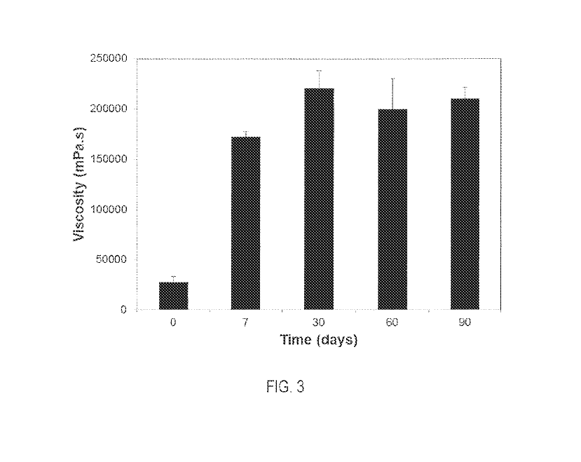

FIG. 3 presents an example of maturation process. The evolution of the composition viscosity over time is exposed. Due to the complex physicochemical processes occurring during maturation (ion and mineral exchange, swelling, hydrogen bonding) the viscosity increased until equilibrium was reached.

### EXAMPLE XVII

### Desired Rheologycal Characteristics; Shear Thinning Behaviour

The desired theological characteristics of the final cosmetic and/or dermocosmetic composition claimed in the invention considers the shear thinning behavior as first characteristic. Because the composition is isolated in a double or more chamber aluminum monoblock container it's necessary that the viscosity of the composition during dosage decreases to allow container empty. Moreover, in a non-limiting example of the present invention the technology used as double chamber aluminum monoblock container is a bag-on-valve technology, in which the composition is injected thought a valve. As consequence, it's also necessary that the viscosity of the composition decreases during container filling,

The shear thinning behavior of the composition of Example I and other similar compositions are presented on FIG. 4. The viscosity of the compositions decreased as shear rate increased. Shear rate was increased in both product filling and closing processes: because the product was pumped thought the small hole of the valve strain rate was increased in this point. This leaded to the decrease of composition viscosity, making the filling/dosing easier. The shear thinning behavior occurs because under a strain the interactions between clay platelets is broken.

### EXAMPLE XVIII

### Desired Rheologycal Characteristics: Thixotropic Behaviour

The desired rheological characteristics of the final cosmetic and/or dermocosmetic composition claimed in the invention considers the thixotropy as second characteristic. Because the present composition is isolated in a double (or more) chamber aluminum monoblock container it's necessary that the viscosity of the products during dosage decreases, but its also necessary that the product recovers its original viscosity in order to avoid liquid draining through the skin, scalp and/or hair and to obtain a good spreading.

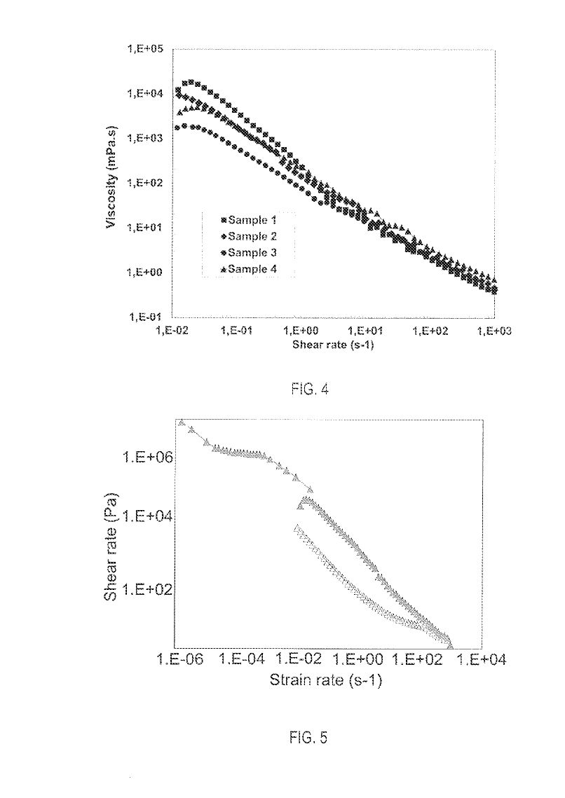

The thixotropic behavior of the composition of Example I and other similar compositions are presented on FIG. 5. In order to analyze the thixotropic behavior a stress-strain experiment was carried out. In these experiments the sample is continuously deformed under different strain rates and the stress is measured.

The experiment is carried out in both directions; first the strain is increased to led the sample deform, the viscosity decreases because the sample has shear thinning behavior. In a second step, the strain is decreased. If the sample is thixotropic the sample reaches its original state once the strain is decreased/eliminated. The compositions claimed in the present invention are thixotropic as they reach their original state once the strain is removed.

The compositions of examples based on the previous art (Example V and VI) didn't have those rheological behaviors. In case of the composition of Example V the viscosity wasn't almost affected by the shear rate as it was mostly composed by water (clay was used as charge, at low concentration) and it behaved almost as a Newtonian fluid. The composition of Example VI had also a shear thinning behavior, as viscosity decreased with strain rate. Nevertheless, once the strain disappeared, the fluid was not able to reach its original viscosity because the clay concentration was not enough for reestablish the original clay platelet conformation.

### EXAMPLE XIX

### Hydrating Property of a Cosmetic Composition in Spray Using a Mixture of White and Red Clays as Active Compounds

Clays are active compounds that usually dry the skin. The composition claimed in the present invention has a characteristic feature of being hydrating/moisturizing.

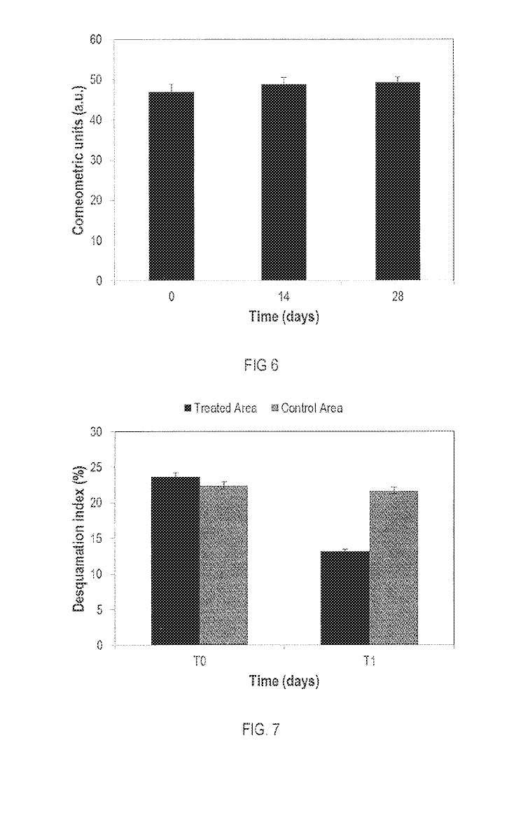

The evaluation of the hydrating/moisturizing properties was carried out with a clinical-instrumental study. The measure was based on the moisturizing index by Internationally recognized CORNEOMETER® which is based on the completely different dielectric constant of water (81) and other substances (mostly <7). The results are present in FIG. 6.

It was observed that the product maintained the skin moisturizing index after 14 and 28 days, indicating a good hydrating or moisturizing behavior is not obvious even for a skilled artisan the obtaining of hydrating masks using high clay concentrations, as is well known in previous art that clay dries the skin, scalp and/or hair. The present invention claims a mask composition containing clay and/or cocoa powder, sodium polyacrylate and natural mineral water that has a hydrating effect, thanks to the synergistic effect of sodium polyamilate.

### EXAMPLE XX

### Exfoliating Efficacy of a Cosmetic Composition in Spray Using White Clay as Active Compound and Natural Mineral Spring Water as Continuous Media

The exfoliation efficiency of the final cosmetic composition in spray containing white clay as active compound was tested in an in vivo experiment. Exfoliation process leads to the improvement on skin elasticity.

The efficiency test was carried out by means of an instrumental study performed on 20 female subjects showing normal to scaly skin (skin with desquamation tendency) on their faces. The subjects were selected according the following inclusion criteria; all the subjects were healthy Caucasian type females older than 18 years old with normal to scaly skins in the absence of any disease. None of those subjects were pregnant or breastfeeding women, people that showed allergies or sensitivity to cosmetic products, toiletries, sunscreens and/or topical drugs, women with dermatological problems or people under pharmacological treatments (both, locally or systematically).

The efficacy of the cosmetic product in spray was evaluated by a non-invasive skin bioengineering technique able to evaluate the exfoliating effect.

A single application of the product was foreseen and the controls were carried out before and after 30 minutes from application. The product was applied under the supervision of an expert dermatologist who evaluated the physiological skin conditions (T0) of each subject. One hemi face of the subjects was treated with the test product applying, on the wet skin, a thin layer of cosmetic product using the hands and with a gentle massage. Eye and mouth area were avoided. The mask was allowed to act until dry, approximately 10 minutes and the product was finally removed using warm wet towels. The other hemi face remained untreated and acted as a positive control.

The desquamation index, an experiment that indicates exfoliation efficiency, was evaluated by means of image analysis (Visioscan VC98 and Corneofix F-20 Courage+Khazaka, Electronid GmgH). The parameter correlates with the extension and the severity of the skin desquamation. Corneofix® tapefoils allowed taking corneocytes from the stratum corneum. The images of the corneocytes stuck to the foil were taken using a Visoscan VC98 camera. The image analysis software evaluated the number, the size and the area occupied by the corneocytes, analyzing the gray level histogram inside the image.

The desquamation index was calculates as:

ID=[2A+(T1×(1−1)+T2×(2−1)+ . . . +Tn×(n−1)]/(n+1)

where: A=total area covered with corneocytes in %; n=thickness of the corneocyte layer; Tn=Sum of the percentage of the single thickness layers,

The average data obtained at each experimental time are presented in FIG. 7.

It was observed that the use of the cosmetic composition for external administration in spray form prepared using white clay as active compound resulted in a statistically significant reduction of corneocytes adhered to the Corneofix® tape. In fact, the desquamation index of the skin decreased after 30 minutes of application of the composition and just with a single use. This reduction was statistically superior to the one in the control area, evaluated after rinsing the skin with water.

Taking into account the results provided, it was concluded that the cosmetic composition in spray containing white clay as active compound showed exfoliating effect that helped to accelerate cellular turnover in the epidermis. The single used of the product determined a reduction of corneocytes adhered to the tape, favoring in this way, the separation of the surface layers of the epidermis (dead cells). Those results indicated improvements on skin elasticity.

### EXAMPLE XXI

### Sebo-Regulating Efficacy of a Cosmetic Composition in Spray Using a Mixture of White and Red Clays as Active Compounds and Natural Mineral Spring Water as Continuous Media

The efficacy of the cosmetic composition for external administration in spray form was tested by means of en in vivo evaluation of the sebo-regulating efficacy. The aim of the study was to evaluate the sebo-regulating efficacy of the cosmetic product in spray containing white clay and red clay as active components on the sebum secretion after repeated application under normal conditions of use. The skin tolerability of the product was also evaluated.

To this end, a clinical-instrumental study was carried out in accordance with the “In Use test” method on 20 female subjects who showed signs of oily skin and no more than twenty-five inflamed and non-inflamed lesions (blackheads, papules and/or pimples) on their faces. The volunteers applied the test product twice a week and also used a base cream and a neutral cleanser during the experimental period. Prior to apply the product, the make-up was removed and the face was cleaned. After wet the face with natural miner water the cosmetic product in spray was dispersed or applied using a gently massage in the area to be treated (half face of each of the volunteers). After the product was dried (at about 10 minutes) the dry clay was washed using wet towels. Finally, the face was again moisturized using natural mineral water and a base cream.

The subjects were selected according the following inclusion criteria; all the subjects were healthy Caucasian type females older than 18 years old with slight to pronounced oily skin showing no more than 25 inflamed and non-inflamed lesions (blackheads, papules and/or pimples) with in the absence of dermatopathies. None of those subjects were pregnant or breastfeeding women, people that showed allergies or sensitivity to cosmetic products, toiletries, sunscreens and/or topical drugs, women with dermatological problems or people under pharmacological treatments (both, locally or systematically).

Evaluations were performed after 14 and 28 days of product use.

The dermatologist in charge of the experimental evaluation analyzed the skin tolerability of the treatment and no adverse skin reactions were monitored indicating a good safety of the cosmetic composition in spray formulated with white clay and red clay as active compounds.

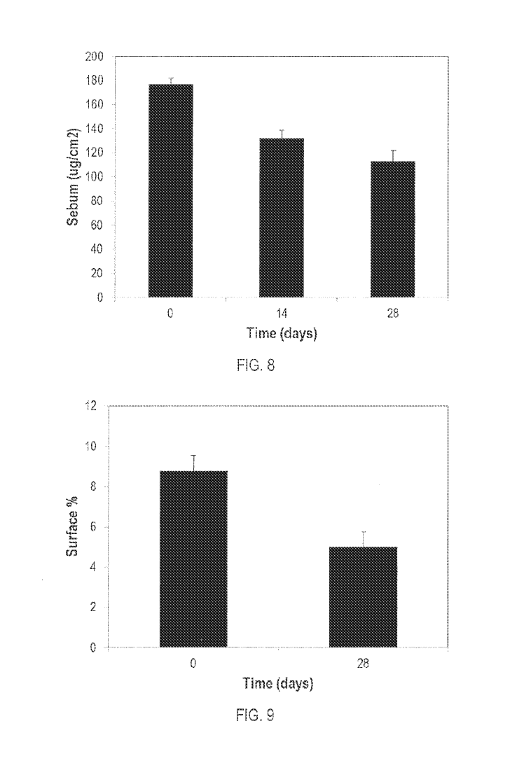

The sebum measurement on the skin was based on the internationally recognized Sebumeter® method. The measurement principle is a photometric method and is carried out using the grease spot photometer. In the method, skin sebum is collected on a 64 mm² synthetic tape contained in a cassette. Afterwards, the measuring head of the cassette is inserted into the aperture of the device, where a photocell measures the transparency. The light transmission represents the sebum content on the surface of the measuring area. A microprocessor calculates the result, which is shown on the display in μg sebum/cm² of the skin. The results achieved are presented in FIG. 8. A significant decrease on the skin sebum was observed first after 14 days and second after 28 days. The results indicated therefore a good sebo-regulator effect of the cosmetic composition.

Additionally, the activity of sebaceous glands on the forehead was evaluated using a Sebufix® F 16. The parameter analyzed is the Pore Surface, defined as the Surface % of spots with size in the range 0-2000. This parameter allows evaluating the percentage surface occupied by spots of size in the range 0-2000 μm.

It was demonstrated that the application of the cosmetic product significantly decreased the percentage of surface occupied by oily spots after 28 days of use, indicating once again a good sebo-regulating property (FIG. 9).

Finally, an in vivo test analyzed if the cosmetic product was or not cornedogenic. Cornedons are inflammations of acne.

The product is classified as non-cornedogenic if it leads to a percentage of variation in the number of lesions (per person) of less than 50% (O. H. Jr. Mills, A. M. Kligman, A human model for assessing cornedogenic substances, Arch. Dermatol., 118, 903-905, 1982). The cornedogenic potential can be classified based on the number of volunteers that manifest a significant increase of lesions on the face, throughout the test period. The evaluation criteria were the following ones: if none of the subjects presented any increase of lesions the product was considered to have low cornedogenic potential; if to 2 subjects out of 10 presented any lesions, the product was middle cornedogenic potential and finally more than 2 subjects out of 10 presented an increase in their lesions, the product was considered to have high cornedogenic potential.

The biweekly use of the product over 4 weeks, in the enrolled volunteers and under the test conditions, did not involve the appearance of lesions.

Therefore, it was concluded that the cosmetic composition in spray formulated with white clay and red clay as active ingredients was a skin care product that showed a sebo-regulator effect by decreasing the skin surface sebum and reducing the percentage of surface covered by oily spots.

The product also maintained skin moisture level after 28 days of use and it was well tolerated by the subjects participating in the study. Moreover, the product may also be classified as non-cornedogenic (low cornedogenic potential).

1) A liquid cosmetic and/or dermocosmetic composition for external administration using a spray/aerosol system comprising:
a) 20 to 80% by weight, based on the total weight of the composition, of at least an active compound consisting of clay and cocoa powder, alone or in combination,
b) 0.001 to 20% by weight, based on the total weight of the composition, of sodium polyacrylate, and
c) 19.997 to 79.997% by weight, based on the total weight of the composition, of a continuous external dispersing phase consisting of natural mineral spring water and wine, alone or in combination.

2) The liquid cosmetic and/or dermocosmetic composition according to claim 1, further comprising at least one skin beneficial compound in a dermatologically effective amount.

3) The liquid cosmetic and/or dermocosmetic composition according to claim 1, further comprising:
a) 0.001 to 20% by weight, based on the total weight of the composition, of sodium polyphosphate,
b) 0.001 to 5% by weight, based on the total weight of the composition, of citric acid.

4) The liquid cosmetic and/or dermocosmetic composition according to claim 1, wherein the concentration of clay used in the composition is chosen in function of the structural, physicochemical and crystallographic properties of the selected clay.

5) The liquid cosmetic and/or dermocosmetic composition according to claim 1, wherein the clay is selected from any of the clay families or groups; kaolinite, smectite/montmorillonite, chlorite and/or mica and mixtures thereof.

6) The liquid cosmetic and/or dermocosmetic according to claim 1, wherein the concentration of cocoa powder used in the composition is chosen in function of the physicochemical properties of the selected cocoa powder.

7) The liquid cosmetic and/or dermocosmetic composition according to claim 1, wherein the composition is provided in an aluminium monoblock container having two or more chambers, and wherein the composition is contained in a chamber without contact with the propellant that is contained in a separate chamber.

8) The liquid cosmetic and/or dermocosmetic composition according to claim 1, wherein the composition suffers a maturation process.

9) The liquid cosmetic and/or dermocosmetic composition according to claim 8, wherein the maturation process of the composition is a mixture of physicochemical processes.

10) The liquid cosmetic and/or dermocosmetic composition according to claim 1, wherein the composition presents specific rheological characteristics that allow the filling and emptying of the composition from the aluminium monoblock container having two or more chambers and the spreading of the liquid without draining.

11) The liquid cosmetic and/or dermocosmetic composition according to claim 1 that is intended to be applied on the skin, scalp and/or hair.

12) The liquid cosmetic and/or dermocosmetic composition according to claim 1, wherein the composition has a hydrating/moisturizing effect to the skin.

13) A liquid cosmetic and/or dermocosmetic composition for external administration using a spray/aerosol system comprising:
a) 20 to 80% by weight, based on the total weight of the composition, of at least an active compound consisting of clay and cocoa powder, alone or in combination,
b) 19.997 to 79.997% by weight, based on the total weight of the composition, of a continuous external dispersing phase consisting of natural mineral spring water and wine, alone or in combination, and
c) at least one skin beneficial compound in a dermatologically effective amount.

14) The liquid cosmetic and/or dermocosmetic composition for external administration using a spray/aerosol system according to claim 1, wherein the wine used is red wine.

15) The liquid cosmetic and/or dermocosmetic composition for external administration using a spray/aerosol system that comprises at least one skin beneficial compound according to claim 2, in which the skin beneficial compound can be a conditioner, a secondary emollient or hydrating agent, a fragrance, a presentative, an anti-microbial compound, a fungicide, a skin conditioner, an absorbent, a hair fixing agent, a soothing compound, an anti-inflammatory compound, a skin lightening or whitening product, an anti-wrinkle product, a compound for acne treatment, a body slimming and any other compound that can be beneficial to skin, alone or in combination.

16) The liquid cosmetic and/or dermocosmetic composition for external administration using a spray/aerosol system according to claim 15, wherein the concentration of the skin beneficial compound is in the interval between 0.01% and 20% by weight.

17) The liquid cosmetic and/or dermocosmetic composition for external administration using a spray/aerosol system according to claim 9, wherein the physicochemical processes involved in the maturation process are ion and mineral exchange (cationic exchange processes), clay and sodium polyacrylate swelling by water and hydrogen bonding between different compounds.
